(19) United States
(12) Patent Application Publication
(10) Pub. No.: US 2008/0004626 A1
(43) Pub. Date: Jan. 3, 2008

(54) Orthopedic coil screw insert

(76) Inventors: Paul A. Glazer, Chestnut Hill, MA (US); Joseph Ting, Acton, MA (US)

Correspondence Address: EDWARDS ANGELL PALMER & DODGE LLP, P.O. BOX 55874, BOSTON, MA, 02205, US

(21) Appl. No.: 11/807,143

(22) Filed: May 25, 2007

Related U.S. Application Data
(60) Provisional application No. 60/808,565, filed on May 26, 2006.

(51) Int. Cl. A61B 17/58

(52) U.S. Cl. 606/73

(57) ABSTRACT

The invention provides an insert for receiving a bone screw. The insert includes a head portion and a body portion extending from the head portion and defined by a helically formed coil. The helically formed coil has an outer diametrical surface configured for engaging a bone and an inner diametrical surface configured for engaging a bone screw extended therethrough. The invention also provides a method of anchoring a bone screw in a bone. The method includes providing an insert for receiving a bone screw, implanting the insert within a bore formed in a bone, and driving a bone screw into the inner diametrical surface of the insert within the bore.

### CROSS-REFERENCE TO RELATED APPLICATION

This application claims the benefit of priority from U.S. Provisional Patent Application Ser. No. 60/808,565, filed May 26, 2006, the disclosure of which is incorporated by reference herein in its entirety.

### BACKGROUND OF THE INVENTION

1. Field of the Invention

The present invention relates to a coil screw insert, and more particularly, to a coil insert for receiving a bone screw to increase the anchoring strength of the bone screw.

2. Description of Related Art

A variety of devices are known in the art for anchoring surgical components to bones. Of such devices, many are directed to bone screws for attaching bones together or to attaching prostheses to bones. There are several uses for bone screws in treating spinal conditions, for example.

Typically, when a bone screw is implemented to treat a bone condition, a drill is used to bore into the bone. The resulting bore typically extends through cortical bone and into cancellous bone. After the bore is formed, a bone screw can be driven into the bore with the threads engaging the bone where possible. Various items can then be anchored to the bone by means of the bone screw in place within the bore.

The anchoring strength of bone screws as described above is limited, however. Anchoring strength can be limited by defects in the bone. For example, osteoporotic bone in older patients is much weaker than bone in young, healthy patients. Also, errors in the placement of the bore can compromise the anchoring strength of bone screws. For example, current orthopedic devices used in the spine rely on screws inserted directly into the vertebral structure. Fixation strength is highly dependent not only on strength and integrity of the bone, but also on the placement of the bores. This is a significant limitation for spinal devices, especially those aimed at non-fusion applications such as dynamic stabilization devices. Placement and insertion of bores and screws is often difficult in bony structures such as the pedicle or facet. An alignment mistake can lead to the bore lying too close to or breaking through the cortex on one side of the bone structure. When a bone is compromised in this manner, it may provide inadequate load distribution for use of a conventional bone screw.

Conventional methods and systems of anchoring structures to bones generally have been considered satisfactory for their intended purpose. However, there remains an ever present need to advance the state of the art for increasing the anchoring strength of structures like bone screws. There also remains a need in the art for a method and a system that can increase the anchoring strength of bone screws in defective bone tissue or compromised bores. The present invention provides a solution for these problems.

### SUMMARY OF THE INVENTION

The subject invention is directed to a new and useful insert for receiving a bone screw. The insert of the invention includes a head portion and a body portion extending from the head portion. The body portion is defined by a helically formed coil. The helically formed coil has an outer diametrical surface configured for engaging a bone and an inner diametrical surface configured for engaging a bone screw extended therethrough.

In accordance with the subject invention, a proximal section of the body portion adjacent the head portion is configured and adapted to conform to and engage with the cortical bone. A distal section of the body portion can be configured and adapted to engage with cancellous bone.

In accordance with another aspect of the invention, the body portion includes means for reducing the diameter of the helical coil. The means for reducing the diameter can be a radially inwardly depending tab formed at a distal end of the coil. The tab can be configured and adapted for engagement by a tool for axially rotating the tab in the direction of the coil to constrict the coil.

In another embodiment of the invention, the insert further includes a sharpened cutting edge winding along at least a portion of the coil adjacent to the outer diametrical surface of the coil. The sharpened edge is configured and adapted to increase fixation strength when the insert engages bone tissue through self-tapping.

In accordance with still another aspect of the invention, the head portion of the insert includes a sleeve affixed to the coil. The sleeve includes a passage therethrough that is generally aligned with the inner diametrical surface of the coil for accommodating a bone screw therethrough. The sleeve can include at least one slot configured and adapted for engagement with a screwdriver for purposes of removal or installation of the insert in a bone. The sleeve can also be configured and adapted to accommodate dynamic compression bone plates.

In another embodiment of the invention, the coil is of a material selected from among titanium alloys, plastics, composites or various grades of stainless steel. The insert can further include a polymer coating disposed on at least a portion of the coil to provide resistance to turning of a bone screw when engaged within the insert. The body portion can be of greater length than the thickness of cortical bone when the insert is engaged within the bone. The coil can be configured and adapted to expand and contract axially as needed to accommodate threads of bone screws of various pitches.

The invention further includes an apparatus for insertion into a bone including a head portion and a body portion extending from the head portion. The body portion is defined by a helically formed coil having an outer diametrical surface configured for engaging a bone and an inner diametrical surface configured for engaging a bone screw extended therethrough. A radially inwardly depending tab is formed at a distal end of the coil for engagement by a tool to reduce the diameter of the coil by twisting. A sharpened cutting edge winds along at least a portion of the coil adjacent to the outer diametrical surface. The sharpened edge is configured and adapted to increase fixation strength when the insert engages bone tissue through self-tapping. The head portion can have a sleeve that is affixed to the coil. The sleeve includes a passage therethrough that is generally aligned with the inner diametrical surface of the coil for accommodating a bone screw therethrough. The apparatus can further include a screw configured and dimensioned to engage the inner diametrical surface of the coil.

The invention also includes a method for anchoring a bone screw in a bone. The method includes the step of providing an insert for receiving a bone screw. The insert has a head portion and a body portion extending from the head portion. The body portion is defined by a helically formed coil having an outer diametrical surface configured for engaging a bone and an inner diametrical surface configured for engaging a bone screw extended therethrough. The method further includes implanting the insert within a bore formed in a bone. The method can include the step of removing the insert from the bore.

In accordance with another aspect of the invention, the method can further include driving a bone screw into the inner diametrical surface of the insert within the bore. The method can also include a step of constricting the coil to facilitate implanting the insert within the bore in the bone. The step of constricting can include applying a tool axially through the coil to engage and twist a radially inwardly depending tab formed at a distal end of the coil to elongate the coil thereby reducing the diameter of the coil for insertion into a bone. The tool can include two portions, one for gripping the head portion of the insert to hold it stationary while a second portion of the tool engages and twists the coil, as described above. The tool can be released controllably from the coil to permit the coil to expand to a rest diameter engaging the walls of the bore in the bone. It is also contemplated that the step of constricting the coil can include constricting the coil within a tubular tool. The coil can be released or expelled from the sleeve into the bore in the bone.

In another embodiment of the invention, the step of implanting includes engaging the bone with the coil by self-tapping, wherein the coil includes a sharpened cutting edge winding along at least a portion of the coil adjacent to the outer diametrical surface. The sharpened edge is configured and adapted to increase fixation strength when the insert engages bone tissue through self-tapping.

These and other features of the insert of the subject invention and the manner of anchoring a bone screw in a bone will become more readily apparent to those having ordinary skill in the art from the following enabling description of the preferred embodiments of the subject invention taken in conjunction with the several drawings described below.

### BRIEF DESCRIPTION OF THE DRAWINGS

So that those skilled in the art to which the subject invention appertains will readily understand how to make and use the insert and method of anchoring a bone screw according to the subject invention without undue experimentation, preferred embodiments thereof will be described in detail hereinbelow with reference to certain Figures, wherein:

FIG. 1a is a perspective view of a representative embodiment of an insert for receiving a bone screw in accordance with the present invention, showing the distal end of the insert;

FIG. 1b is a perspective view of the insert of FIG. 1a in accordance with the present invention, showing the proximal end of the insert having a slotted sleeve;

FIG. 2a is an elevation view of the insert of FIG. 1a in accordance with the present invention, showing the coil in a relaxed state prior to insertion in a bone;

FIG. 2b is an elevation view of the insert of FIG. 1a in accordance with the present invention, showing the coil in a constricted state prior to insertion in a bone;

FIG. 2c is an elevation view of the insert of FIG. 1a in accordance with the present invention, showing the insert within a bore in the bone, with the coil expanded outward against the cortical bone and cancellous bone;

FIG. 2d is a partial cross-sectional elevation view of the insert of FIG. 1a in accordance with the present invention, showing a bone screw anchored within the bore in the bone by means of the insert;

FIG. 3a is an elevation view of the insert of FIG. 1a in accordance with the present invention, showing a narrow piece of bone with a bore formed too close to the edge of the bone for a conventional bone screw;

FIG. 3b is an elevation view of the insert of FIG. 1a in accordance with the present invention, showing the insert implanted within the bore of FIG. 3a to rectify the compromised bone structure by receiving a bone screw and distributing the loads thereon;

FIG. 4a is a chart showing load versus displacement for pull tests on samples with a bone screw in a helical insert implanted in a polyurethane foam block; and

FIG. 4b is a chart showing load versus displacement for pull tests on samples with a bone screw implanted in a polyurethane foam block without a helical insert.

### DETAILED DESCRIPTION OF THE PREFERRED EMBODIMENT

Reference will now be made in detail to the present preferred embodiments of the invention, an example of which is illustrated in the accompanying drawings. The method and corresponding steps of the invention will be described in conjunction with the detailed description of the system.

The devices and methods presented herein may be used for treating bone conditions. The present invention is particularly suited for enhancing the anchoring strength of bone screws, such as when treating spinal conditions.

In accordance with the invention, an insert is provided for receiving a bone screw. The insert includes a head portion and a body portion. The body portion is defined by a helically formed coil, which has an outer diametrical surface configured for engaging a bone and an inner diametrical surface configured for engaging a bone screw extended therethrough.

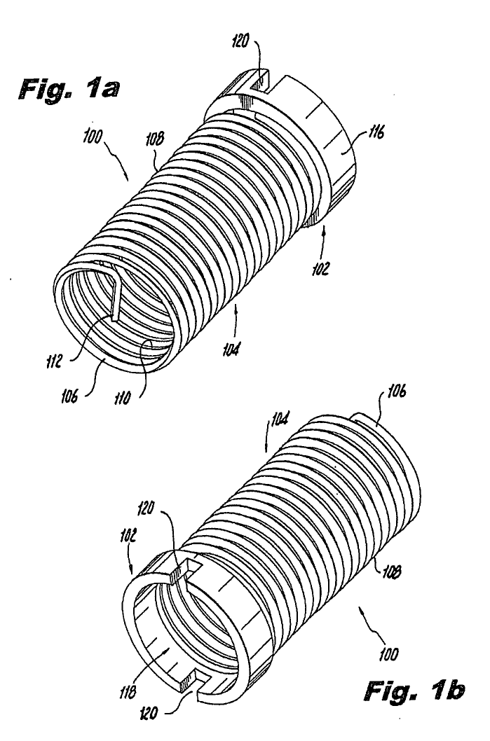

Referring now to the drawings wherein like reference numerals identify similar features or elements of the various embodiments of the subject invention disclosed herein, there is illustrated an insert for receiving a bone screw. For purposes of explanation and illustration, and not limitation, a partial view of an exemplary embodiment of the insert in accordance with the invention is shown in FIG. 1a and is designated generally by reference character 100. Other embodiments of an insert in accordance with the invention, or aspects thereof, are provided in FIGS. 1b-4b, as will be described.

As depicted in FIGS. 1a and 1b, insert 100 is provided having a head portion 102. A body portion 104 extends from head portion 102. Body portion 104 includes a helical coil 106. Coil 106 has an outer diametrical surface 108 and an inner diametrical surface 110. Outer surface 108 is configured and adapted to engage bone tissue and inner surface 110 is configured and adapted to engage a bone screw, as described below.

Coil 106 can be formed in any suitable manner. For example, a straight strip of material can be wound around a mandrel to form coil 106. It is also possible to form coil 106 out of a block of material through machining, etching, electrochemical deposition, or any other suitable process. There are a variety of cross-sectional shapes available for the helically wound member forming coil 106, such as triangular, teardrop shaped, round, circular, diamond shaped, square, rectangular, or any other suitable shape. Moreover, the overall cross section of coil 106 itself as a whole can be circular as depicted, or can be any other suitable shape including non-round shapes such as hexagonal or square. Those skilled in the art will readily appreciate a variety of suitable methods for forming coil 106 that can be used without departing from the spirit and scope of the invention.

There are a variety of suitable materials that can be used for coil 106. Preferably coil 106 is of a highly biocompatible material. Suitable materials include titanium alloys. There are also several varieties of stainless steel known in the art that can be used for coil 106. It is also possible that plastics or composite materials can be used. Those skilled the art will readily appreciate that any suitable material can be used for coil 106 without departing from the spirit and scope of the invention.

With continued reference to FIG. 1a, tab 112 depends from one end of coil 106. Tab 112 is an integral part of coil 106 that is bent radially inward from the rest of coil 106. The function of tab 112 is to provide an attachment point for a tool, such as forceps or other suitable instruments, which can grab tab 112 to axially twist coil 106. Twisting coil 106 can be useful in constricting the diameter of coil 106 down to a size more conducive to insertion in bore, as described below. Tab 112 can be roughened, flattened, or otherwise treated to make it easier to grip with a tool.

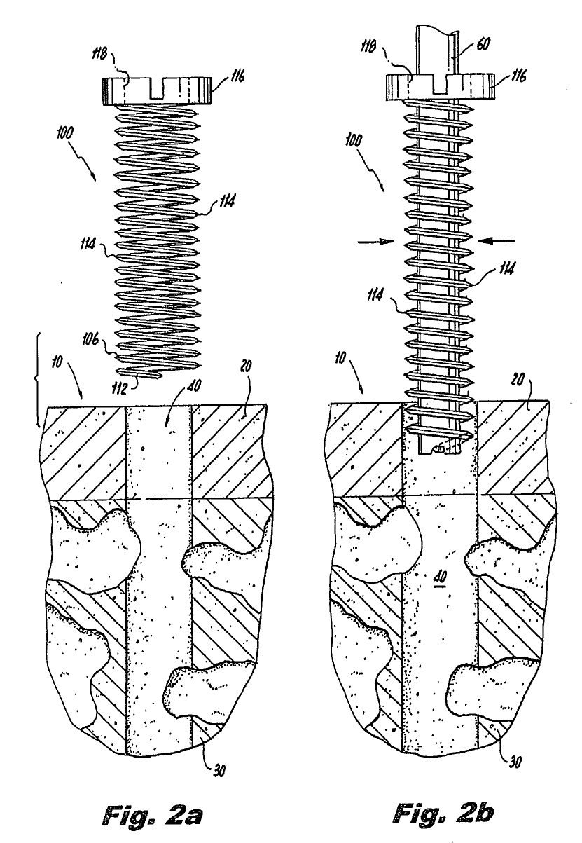

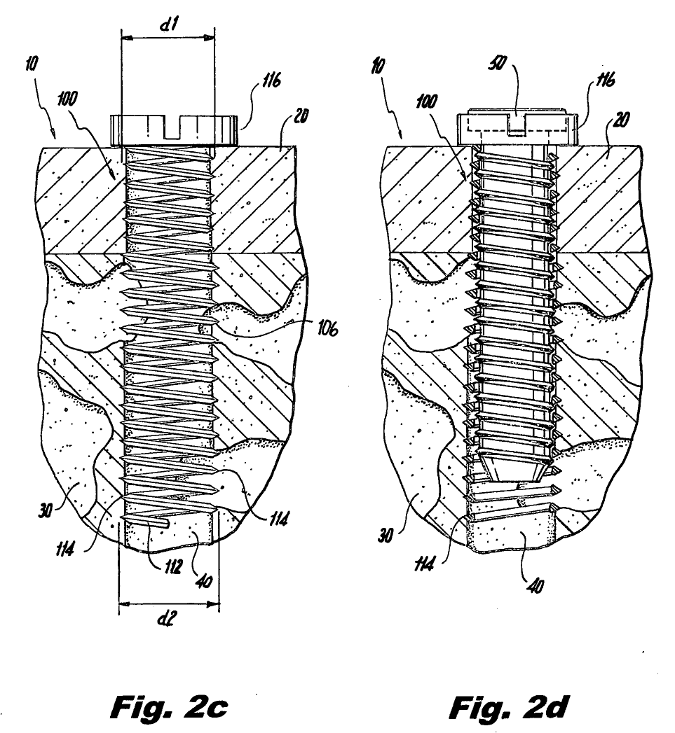

There are other possible means for reducing the diameter of coil 106 for insertion into a bore. For example, a tab like tab 112 could be formed of a separate piece joined to coil 106. Tab 112 could extend across the entire diameter of coil 106 and could even be joined to the other side of coil 106. An elongate tool 60 (shown in FIG. 2b) can be used to engage tab 112 and stretch coil 106 axially, with or without twisting, to reduce the diameter of coil 106. A second tool can be used to grip and hold sleeve 116 stationary while the tool 60 twists and/or stretches coil 106 by means of tab 112. It is also possible that tool 60 itself could include a portion that holds 116 stationary while tool 60 engages tab 112 to constrict coil 106.

Another possible means of reducing the diameter of coil 106 for insertion is a tubular tool, which could be used to house the constricted coil 106 prior to insertion. Housed inside a tubular tool, coil 106 could be ejected into a bone by rotating or pushing it out of the tubular portion of the tool, for example by a rod nested in the tube portion of the tubular tool. Those skilled in the art will readily appreciate other means of constricting coil 106 for insertion that can be used without departing from the spirit and scope of the invention.

In further accordance with the invention, head portion 102 can include sleeve 116, which is attached to coil 106 by conventional metal joining processes, such as welding, laser welding, or spot welding. Sleeve 116 has a passage 118 extending therethrough, which is generally aligned with inner surface 110. Passage 118 allows for a bone screw to pass through sleeve 116 to access inner surface 110. The inner contours of coil 106 can be configured to accommodate the threads of a typical bone screw, such as pedicle or facet screws. Head portion 102 can provide an attachment point for a variety of devices, e.g. rods, plates, or the like.

As best seen in FIG. 1b, sleeve 116 also includes at least one slot 120, which allows for a screwdriver to engage sleeve 116. A screwdriver can thus be used to extract insert 100 from a bone if necessary. Also, it is possible for sleeve 116 to have an outer perimeter that is hexagonal or any other suitable shape to allow gripping with a tool such as a wrench. This can serve a similar function to slot 120, or can be used in lieu thereof. Those skilled in the art will appreciate that sleeve 116 is an optional feature, however it is advantageous to have sleeve 116 in case insert 100 needs to be removed from a bone. If a specialized tool is used to implant insert 100 by means of tab 112, sleeve 116 can allow for removal of insert 100 without requiring the same specialized tool. One advantage of using insert 100 without sleeve 116 is if it is desired to constrict coil 106 in a tubular tool for insertion into a bone, as described above, since there is no added diameter of head portion 102.

FIG. 2a shows insert 100 in an elevation view that reveals the sharpened cutting edges 114 formed along the outer surface 108 of coil 106. Sharpened edge 114 lends insert 100 extra anchoring strength through self-tapping within a bone. Those skilled in the art will appreciate that sharpened edge 114 is an optional feature. It is possible to have a sharpened edge 114 along only one part or multiple discrete portions of coil 106. Sharpened edge 114 can be formed with various grinding processes as is known in the art, either before or after coil 106 is formed. In the case of coil 106 being formed of a straight strip of material wound into a coil 106, if the strip has a sharp edge to begin with, edge 114 can be formed simply by keeping the sharp edge to the outside when winding the strip into coil 106.

In accordance with another aspect of the invention, a method of anchoring a bone screw in a bone is provided. The method includes the step of providing an insert for receiving a bone screw. The insert has a head portion and a body portion extending from the head portion. The body portion of the insert is defined by a helically formed coil, which has an outer diametrical surface configured for engaging a bone and an inner diametrical surface configured for engaging a bone screw extended therethrough. The method further includes the steps of implanting the insert within a bore formed in a bone and driving a bone screw into the inner diametrical surface of the insert within the bore.

For purposes of illustration and not limitation, as embodied herein and as depicted in FIGS. 2a-2d, an insert (e.g. 100) is provided. With continued reference to FIG. 2a, insert 100 is shown in the relaxed, or unconstricted state. This is the state of insert 100 prior to being used to anchor a bone screw in a bone, as will now be described. A bore 40 is preferably drilled or otherwise formed in bone 10, as is known in the art. Bore 40 preferably passes through the layer of cortical bone 20 and well into the cancellous bone 30. It is possible to practice the invention wherein bore 40 is pre-tapped, however this is optional.

With reference now to FIG. 2b, a tool, such as forceps, can be inserted through passage 118 and through coil 106 to grip tab 112 (see FIG. 1a). The tool can then be twisted until coil 106 reaches a constricted state, shown in FIG. 2b, in which the diameter of coil 106 is small enough to be inserted into bore 40. The constricted state is indicated by the large arrows in FIG. 2b.

With coil 106 in the constricted state, insert 100 can be advanced into bore 40 to the desired depth. FIG. 2c shows the full length of coil 106 embedded in bore 40. In this position, the tool holding tab 112 can be used to untwist and relax coil 106. This has the effect of expanding coil 106 outward against the bone tissue of bore 40. Sharpened edge 114 can help coil 106 cut slightly into the bone tissue for increased engagement. Those skilled in the art will appreciate that it is also possible to drive insert 100 into bore 40 like a screw by twisting, for example by a screw driver engaged in slot 120, without departing from the spirit and scope of the invention. In this case, sharpened edge 114 can form a tap as it is wound into bore 40.

Since cortical bone 20 has substantially greater hardness than cancellous bone 30, it is usually not possible for conventional bone screws to maximally grip both bone layers. Conventional bone screws tend to grip strongly in cortical bone 20 and weakly in cancellous bone 30. However, as shown in FIG. 2c, insert 100 expands to meet any bone tissue, be it hard or soft. The portion of coil 106 engaged with cortical bone 20 in the expanded state may be relatively constricted as indicated by small diameter d, in FIG. 2c. The portion of coil 106 in cancellous bone 30 is relatively expanded to a greater diameter than the portion in cortical bone 20, as indicated by diameter d₂, which is slightly larger than d, in FIG. 2c. The threads of conventional bone screws cannot constrict through cortical bone 20 and then expand in cancellous bone 30 in this manner. Thus conventional bone screws cannot gain full purchase in cancellous bone 30 to the extent insert 100 can.

With reference now to FIG. 2d, once insert 100 is firmly in place inside bore 40, a conventional bone screw 50 can be driven through passage 118 of sleeve 116 and into the interior of coil 106. Driving bone screw 50 into insert 100 in this manner provides additional urging of coil 106 against bone 10. Coil 106 can be coated with a polymer or other suitable coating to enhance the grip between coil 106 and bone screw 50, and between coil 106 and bore 40, if appropriate to prevent slipping. Those skilled in the art will appreciate that bone screw 50 and insert 100 in place in bone 10, as shown in FIG. 2d, can be removed by essentially the same steps outlined above, only in reverse order. For example, bone screw 50 can be removed and then a screw driver or other tool could engage sleeve 116 and remove insert 100 by twisting. It is also possible that insert 100 can be removed by re-engaging a tool such as tool 60 to tab 112, constricting coil 106 to a reduced diameter, and then extracting insert 100 from bore 40.

Coil 106 increases the surface area of engagement with bone 10 over that of conventional bone screws alone, and thereby distributes loads throughout bore 40 more favorably. Additionally, coil 106 can expand to conform to cancellous bone 30, including defects and pores within bore 40. For these reasons, insert 100 lends superior anchoring strength when used in conjunction with conventional bone screws.

It is preferable that the length of insert 100 exceeds the thickness of cortical bone 20. However, the invention can be used in situations where cortical bone 20 is thicker than the length of insert 100. Moreover, since insert 100 includes a flexible coil 106, the length of insert 100 can expand or contract in order to conform to a particular bore in a bone or to conform to a bone screw. In this manner, insert 100 can be used with bone screws of a wide range of thread pitches and lengths without modification.

The enhanced anchoring strength provided by insert 100 can benefit a variety of objects needing to be anchored to bones. For example, insert 100 can be used in fusing two bones pieces together. Also, insert 100 can be used to more securely anchor dynamic stabilization devices, e.g. a Dynesys® Dynamic Stabilization System available from Zimmer Spine in Minneapolis, Minn. Those skilled in the art will readily appreciate that sleeve 116 could be modified, for example, to accommodate dynamic or static compression bone plates. Those skilled in the art will further appreciate that with little or no modification, insert 100 can be used to anchor any of a wide variety of implements to a bone without departing from the spirit and scope of the invention.

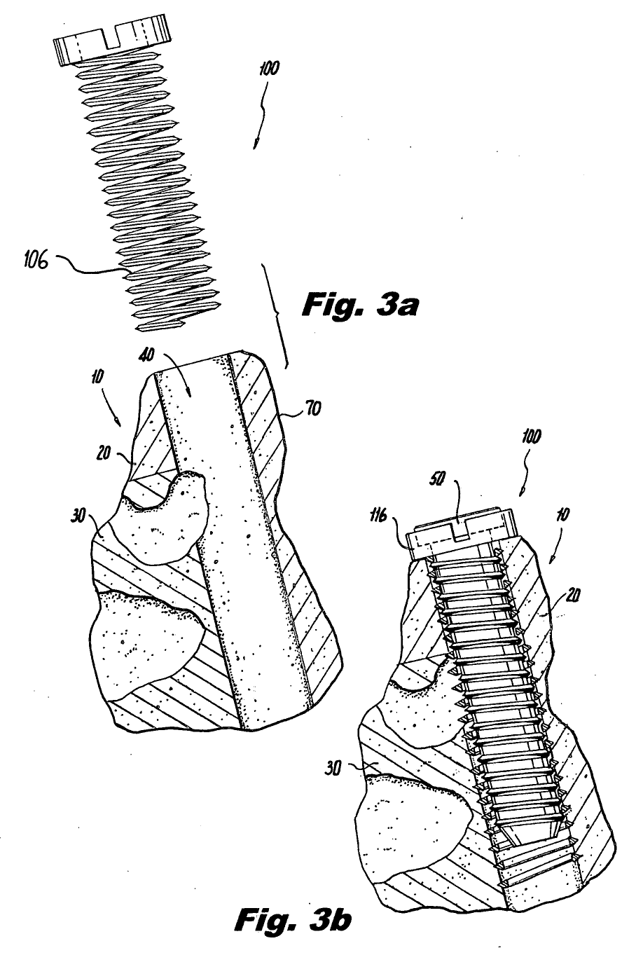

The invention provides enhanced anchoring strength for bone screws extending into relatively soft cancellous bone. Those skilled in the art will appreciate that this is particularly applicable to osteoporotic bone or bone having other structural defects. Another situation in which the invention provides enhanced anchoring strength is depicted in FIGS. 3a and 3b. FIG. 3a shows a bone 10 in which a bore 40 has been drilled with a misalignment. The resulting bore 40 is dangerously close to the surface 70 of bone 10 along most of the length of bore 40. If such a misalignment exists, it is often futile to use a conventional bone screw in the misaligned bore because the weakened condition of the bone will not provide sufficient anchoring strength. The unfavorable load distribution resulting from a conventional bone screw directly engaged within weakened portions of compromised bone 10 precludes use of conventional bone screws in many such situations.

However, utilizing an insert as described above can enhance the anchoring strength of a bone screw in this situation. FIG. 3b shows insert 100 engaged within bore 40. As described above, coil 106 conforms to the bone to a greater extent than bone screws alone. In the situation depicted in FIG. 3b, insert 100 provides for more favorable load distributions within bore 40, which can make it possible to use a bone screw in a misaligned bore after all. Coil 106 helps spread loads from compromised bone to healthy, intact bone. Those skilled in the art will appreciate that this can allow use of misaligned bores in some circumstances that would otherwise be impossible with conventional bone screws alone.

Laboratory tests have confirmed that inserts according to the invention enhance the anchoring strength of conventional bone screws. The tests were conducted under the ASTM F 543-02 standard. Bone screws with and without inserts were implanted in polyurethane foam blocks. Pilot holes with a diameter of 4.95 mm (0.19 in) were drilled into the foam block. A first set of foam blocks received a conventional bone screw in the pilot hole, while a second set of foam blocks received a helical insert and bone screw. An Instron® 3344 tension test apparatus was used to load the bone screws in tension within the foam blocks until failure or release from the foam block. The test apparatus pulled the bone screws at a speed of 5 mm/min (0.20 in/min).

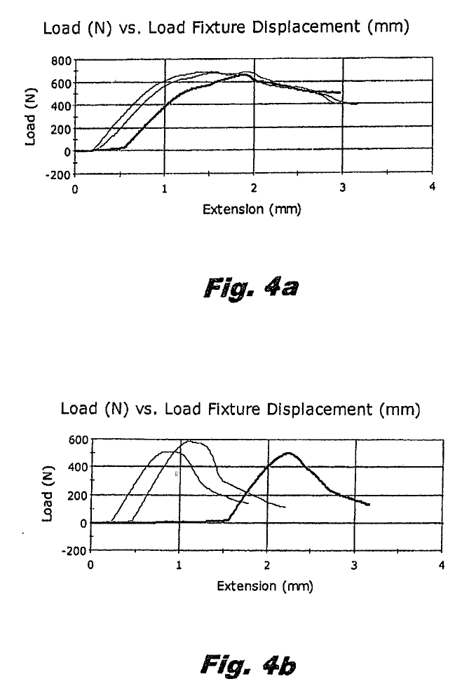

Results from the experiments are shown in FIGS. 4a-4b. FIG. 4a shows a plot of Load (N) versus extension (mm) for tests on three specimens having 6.5 mm (0.25 in) diameter bone screws in helical inserts 12 mm (0.47 in) long. The average peak load was approximately 678 N (152 lbf). FIG. 4b shows a similar plot for three specimens having 6.5 mm (0.25 in) diameter bone screws without helical inserts. In this case, the average peak load is only about 533 N (120 lbf). The substantial difference in average peak load suggests the effectiveness of the helical inserts in anchoring bone screws.

While the apparatus and methods of the subject invention have been shown and described with reference to preferred embodiments, those skilled in the art will readily appreciate that various changes and/or modifications may be made thereto without departing from the spirit and scope of the subject invention as defined by the appended claims.

What is claimed is:

1. An insert for receiving a bone screw comprising:
a) a head portion; and
b) a body portion extending from the head portion and defined by a helically formed coil, the helically formed coil having an outer diametrical surface configured for engaging a bone and an inner diametrical surface configured for engaging a bone screw extended therethrough.

2. An insert as recited in claim 1, wherein a proximal section of the body portion adjacent the head portion is configured and adapted to conform to and engage with cortical bone.

3. An insert as recited in claim 2, wherein a distal section of the body portion is configured and adapted to engage with cancellous bone.

4. An insert as recited in claim 1, wherein the body portion includes means for reducing the diameter of the helical coil.

5. An insert as recited in claim 4, wherein the means for reducing the diameter is a radially inwardly depending tab formed at a distal end of the coil.

6. An insert as recited in claim 5, wherein the tab is configured and adapted for engagement by a tool for axially rotating the tab in the direction of the coil to constrict the coil.

7. An insert as recited in claim 1, further comprising a sharpened cutting edge winding along at least a portion of the coil adjacent to the outer diametrical surface of the coil, the sharpened edge being configured and adapted to increase fixation strength when the insert engages bone tissue through self-tapping.

8. An insert as recited in claim 1, wherein the head portion of the insert includes a sleeve affixed to the coil, the sleeve including a passage therethrough that is generally aligned with the inner diametrical surface of the coil for accommodating a bone screw therethrough.

9. An insert as recited in claim 8, wherein the sleeve includes at least one slot configured and adapted for engagement with a screwdriver for purposes of removal or installation of the insert in a bone.

10. An insert as recited in claim 8, wherein the sleeve is configured and adapted to accommodate dynamic compression bone plates.

11. An insert as recited in claim 1, wherein the coil is of a material selected from a list consisting of: titanium alloys, plastics, composites, and stainless steel.

12. An insert as recited in claim 11, further comprising a polymer coating disposed on at least a portion of the coil to provide resistance to turning of a bone screw when engaged within the insert.

13. An insert as recited in claim 1, wherein the body portion is configured and adapted to be of greater length than the thickness of cortical bone when the insert is engaged within the bone.

14. An insert as recited in claim 1, wherein the coil is configured and adapted to expand and contract axially as needed to accommodate threads of bone screws of various pitches.

15. A method of anchoring a bone screw in a bone, the method comprising:
a) providing an insert for receiving a bone screw, the insert having:
i) a head portion; and
ii) a body portion extending from the head portion and defined by a helically formed coil, the helically formed coil having an outer diametrical surface configured for engaging a bone and an inner diametrical surface configured for engaging a bone screw extended therethrough; and
b) implanting the insert within a bore formed in a bone.

16. A method as recited in claim 15, further comprising a step of driving a bone screw into the inner diametrical surface of the insert within the bore.

17. A method as recited in claim 16, further comprising a step of constricting the coil to facilitate implanting the insert within the bore in the bone.

18. A method as recited in claim 17, wherein the step of constricting the coil includes applying a tool to engage and twist a radially inwardly depending tab formed at a distal end of the coil.

19. A method as recited in claim 17, wherein the step of constricting the coil includes constricting the coil within a sleeve of a tool, wherein the coil can be expelled from the sleeve into the bore in the bone.

20. A method as recited in claim 17, wherein the step of implanting includes engaging the bone with the coil by self-tapping, wherein the coil includes a sharpened cutting edge winding along at least a portion of the coil adjacent to the outer diametrical surface, the sharpened edge being configured and adapted to increase fixation strength when the insert engages bone tissue through self-tapping.

21. A method as recited in claim 15, further comprising a step of removing the insert from the bone.

22. An apparatus for insertion into a bone comprising:
a) a head portion;
b) a body portion extending from the head portion and defined by a helically formed coil, the helically formed coil having an outer diametrical surface configured for engaging a bone and an inner diametrical surface configured for engaging a bone screw extended therethrough;
c) a radially inwardly depending tab formed at a distal end of the coil for engagement by a tool to reduce the diameter of the coil by twisting;
d) a sharpened cutting edge winding along at least a portion of the coil adjacent to the outer diametrical surface, the sharpened edge being configured and adapted to increase fixation strength when the insert engages bone tissue through self-tapping; and
e) a sleeve affixed to the coil, the sleeve including a passage therethrough that is generally aligned with the inner diametrical surface of the coil for accommodating a bone screw therethrough.

23. An apparatus as recited in claim 22, further comprising a screw configured and dimensioned to engage the inner diametrical surface of the coil.
